(19) United States
(12) Patent Application Publication
(10) Pub. No.: US 2015/0320616 A1
(43) Pub. Date: Nov. 12, 2015

(54) ABSORBANT ARTICLE WITH ABSORBANT STRUCTURE WITH CROSS-DIRECTIONALLY ORIENTED PASSAGEWAY

(71) Applicant: CONCEPTS FOR SUCCESS (c4s), Euskirchen (DE)

(72) Inventor: Christoph Schmitz, Euskirchen (DE)

(73) Assignee: Christoph Schmitz, Euskirchen (DE)

(21) Appl. No.: 14/649,918

(22) PCT Filed: Dec. 4, 2013
(86) PCT No.: PCT/EP2013/075448

§ 371 Date: Jun. 5, 2015

(30) Foreign Application Priority Data
Dec. 5, 2012 (GB) 1221882.2

(51) Int. Cl. A61F 13/539

(52) U.S. Cl. CPC A61F 13/539; A61F 2013/530883

(57) ABSTRACT

The present invention is an absorbent article comprising at least one predominantly cross-directionally extending discontinuity, such as a slit, by which the absorbent core of the structure is adapted to form a z- and y-directionally opened passageway.

### FIELD OF THE INVENTION

The present invention relates to absorbent articles, in particular to absorbent articles designed for receiving bodily exudates such as urine, menses, or faecal material, such as adult incontinence articles or baby diapers, and more particular to articles which exhibit a three-dimensional shape thereby creating a passageway for bodily exudates.

### BACKGROUND

Absorbent articles for use in hygienic applications are well known in the art. Also known in the art are approaches for adapting the article shape better to the contours of the body of a wearer. One of these approaches introduces longitudinally oriented bulges in the article, such as described in WO99/25300A1, aiming at convexo-concavely shaped articles, especially, adult incontinence articles. Also in EP0850628A1 a hump or bulge is combined with a trough like shaped region, particularly adapted for feminine care articles. WO2006/109274A1 describes an absorbent article with a particular arrangement of leg hoops and a cup-like shape. WO2011/064275A1 describes a similar approach, wherein the longitudinal side margins of the article are overfolded and tacked down at least in the crotch region. Also described therein is a faeces separation element in the form of a secondary topsheet aiming at keeping faeces away from the skin of a wearer. It is also known to provide selected cut or separation lines aiming at improving fit and/or liquid handling of the article. U.S. Pat. No. 3,848,599 describes articles wherein segments of the absorbent core may be placed in a shingled arrangement between pleated top- and backsheet. As the pleats are affixed at the side margins, an extension along the longitudinal centre line will create a certain cup shape in the article. US2005/0124953 describes an absorbent core with a spacing along the longitudinal centre line, allowing cross-directional deformation and henceforth aiming at improved fit. Similarly, WO2010/070577 describes a longitudinally extending spacing in a core element, whilst a secondary core element may be positioned over this spacing.

However, there is still a need for further adapting the conformability of absorbent articles without impeding the liquid handling properties. There is also a need for further improving the separation between solid and fluid exudates in absorbent articles and to keep exudates away from wearer's skin.

### SUMMARY

The present invention is an absorbent article for being worn on the lower torso of a wearer, which comprises
a) a topsheet layer adapted to be in contact with a wearer during its intended use;
b) a backsheet layer positioned opposite to the topsheet away from a wearer during its intended use;
c) an absorbent structure enveloped there between, comprising a front, rear and crotch region thereby defining a longitudinal orientation of the article and the structure and a cross-directional orientation perpendicular thereto. The article further comprises a cross-directionally extending crotch line for being positioned in the crotch of the wearer;
d) two predominantly longitudinally extending side margins, which are folded over at least in a portion of the crotch region;
e) a pick-up attachment for attaching the overfolded portions of the topsheet at least in a portion of the crotch region to the topsheet;

In the crotch region of the article,
i) the absorbent structure comprises a first discontinuity exhibiting a length projection onto the cross-directional centre line, which is larger than zero, separating in the longitudinal direction a first and a second centre core section along a discontinuity line.
ii) at least a portion of the first centre core section is attached to the topsheet-layer and essentially unattached to the backsheet layer at least along the longitudinal centre line, and
iii) at least a portion of the second centre core section is attached to the backsheet layer and essentially unattached to the topsheet layer at least along the longitudinal centre line.

Thus, in an in use configuration the topsheet layer and the first centre core section attached thereto is lifted towards the wearer whilst the backsheet layer and the second centre core section attached thereto are pulled into the opposite direction, such that the discontinuity forms a passageway for allowing bodily exudates to pass through. Optionally the discontinuity may extend over the full cross-directional width of the absorbent structure. In an alternative to this option, the discontinuity does not extend into the margins of the absorbent structure, and the cross-directional extension of the discontinuity and the respective attachment respectively unattachment regions is at least 1 cm, preferably at least 2 cm, but less than 100% of the cross-directional width of the absorbent structure in the region of the discontinuity.

The article may further comprise at least a further discontinuity, not extending into the side margins of the absorbent structure or not extending into the first discontinuity.

In the case of comprising at least two further discontinuities, these are preferably essentially symmetrical to the longitudinal centre line of the article and may optionally extend into each other. Any of the further discontinuities exhibits preferably a length projection onto the cross-directional centre line, which is larger than zero.

### BRIEF DESCRIPTION OF THE FIGURES

FIG. 1 A to H show schematically the principles of an execution of the present invention.

FIGS. 2 A and B show schematically another execution of the present invention.

FIG. 3A to 3C show schematically a further execution according to the present invention.

FIG. 4 shows schematically further options for positioning discontinuities in the product.

Same numerals in various figures refer to same features or elements.

### DETAILED DESCRIPTION

The present invention is an absorbent article for being worn on the lower torso of a wearer, such as a disposable absorbent article like a baby diaper or an adult incontinence article, or a feminine hygiene article. The article may be a pants-style article, or an open article with closure means such as adhesive tapes or mechanical fastener, or a hybrid article, such as an article, which is delivered pre-closed, but which can be opened and reclosed by a user or caretaker.

Corresponding to the body portions of a wearer, the article comprises a front region and rear region and a crotch region there between, whereby a longitudinal extension is defined as front-to-back and a cross-directional extension perpendicularly thereto (i.e. along a left-right direction). The article is typically mirror symmetric along its longitudinally extending centre line. Often, though not necessarily, such a product is designed and worn such that it terminates at the same height in the front and in the rear, such that cross-directional middle line is positioned such that it corresponds to the crotch line on a wearer, which extends in a left-right orientation in the middle of the crotch region of a standing wearer. Typically, the article is not symmetric to this middle line. Perpendicularly to both the longitudinal and the cross-directional extension, the article exhibits a z-directional or thickness extension.

The article comprises margins, such as predominantly longitudinally extending ones, also referred to as side margins, and predominantly cross-directionally extending ones, also referred to as front and rear margins.

Within the present context of lines or margins, the term “predominantly” refers to the fact, that a straight or curvilinear line, which has a projected longitudinal length, i.e. a length determined by projecting the line to the longitudinal centre line, which is larger than its projected cross-directional length, extends “predominantly” in the longitudinal direction—and vice versa. If the projected longitudinal length is equal to the projected cross-directional length, it is considered to be predominantly longitudinally extending.

These directions and extensions apply primarily to an article in its in-use configuration. A skilled person will readily realize that then the longitudinal centre line has approximately an U-shape, and that for example in the waist region of a wearer the width direction will correspond to the waist circumference. Accordingly, the z-direction corresponds to the thickness of the article. The term “up” or “upwardly” refers to a direction towards a wearer during use, “down” or “downwardly” away from the wearer.

Also when the article is in its pre-use configuration, such as when being manufactured or taken out of a package, any of these directions may be quite readily determined. Even if an article or parts thereof are folded, the skilled person will readily identify the portions and how these correspond to the regions of a wearer.

Such articles comprise typically an absorbent structure, often also referred to as absorbent core, which may be made of a single, unitary element, or which may comprise several elements, and which is enveloped between a topsheet layer and a backsheet layer. The topsheet layer is intended to be in contact with the skin of the wearer, though not necessarily all parts of the topsheet layer will contact the skin, but may be just oriented towards the skin, or may overlay other parts of the article. Accordingly, the opposite backsheet layer may not everywhere be positioned away from the skin of the wearer, but may actually in some smaller parts of the article contact the skin.

The term “layer” in the context of topsheet and backsheet refers to the fact, that the surface of the article is formed by these layers. Other intermediate materials or layers may be positioned between the topsheet layer and the core and/or the topsheet layer and the backsheet layer (e.g. in the enveloping regions surrounding the core).

The present invention provides an improved way of handling of bodily exudates, such as urine, faeces, or menses, which shall be most efficiently kept away from the skin of the wearer, thereby accounting for the different positioning of exudates emanating body orifices or organs as well as for the different consistency of the exudates and potential detrimental effects of e.g. having faeces and urine contacting each other. Also, the present invention allows for guiding e.g. urine away from wearers skin, even in case of unfavourable positioning of a wearer. As to the latter, current articles often fail to address good urine handling in particular when a female wearer is in a reclined position.

The articles according to the present invention have overfolded longitudinally extending side margins at least in a portion of the crotch region at least in a pre-use configuration.

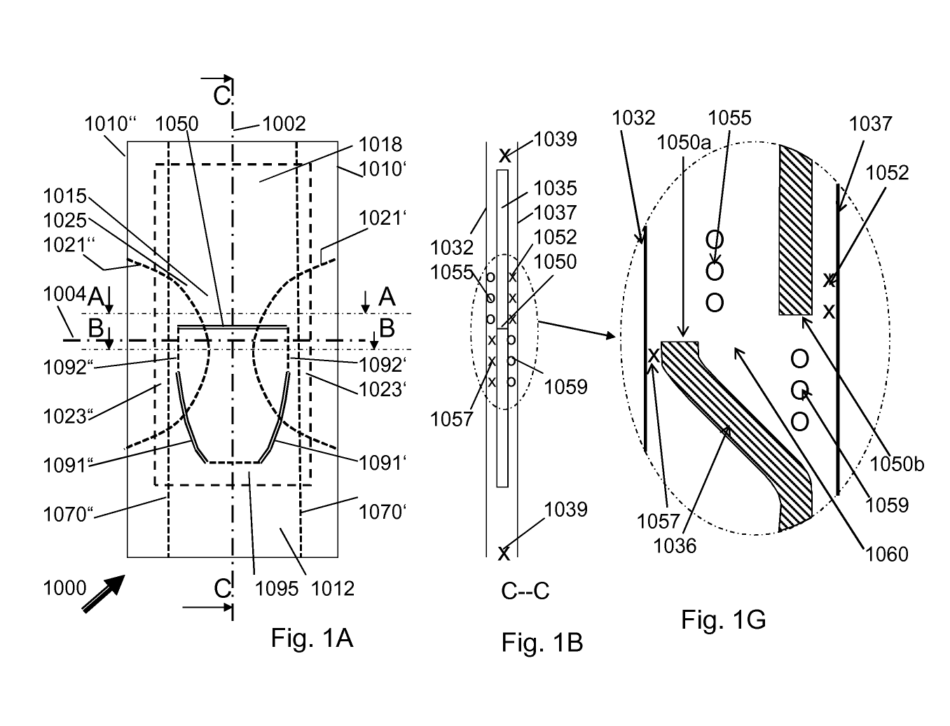

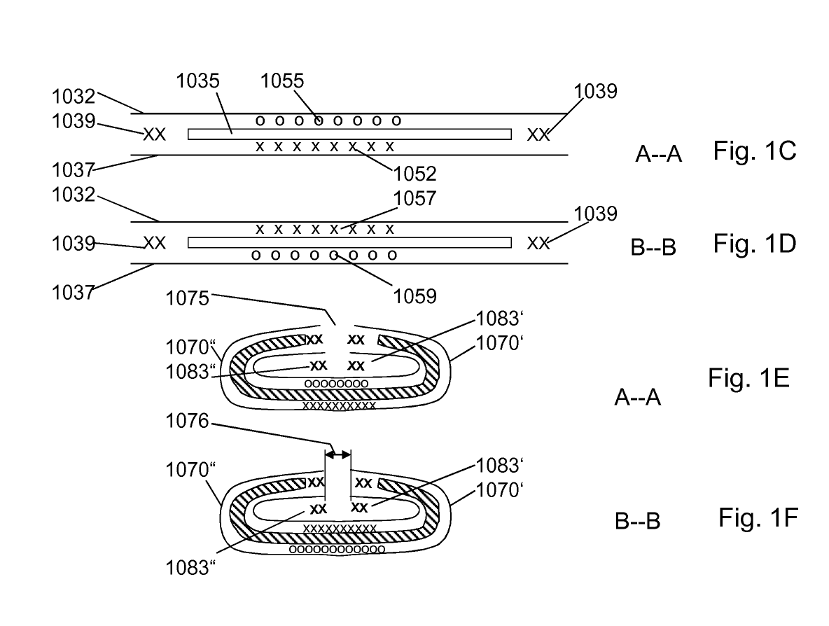

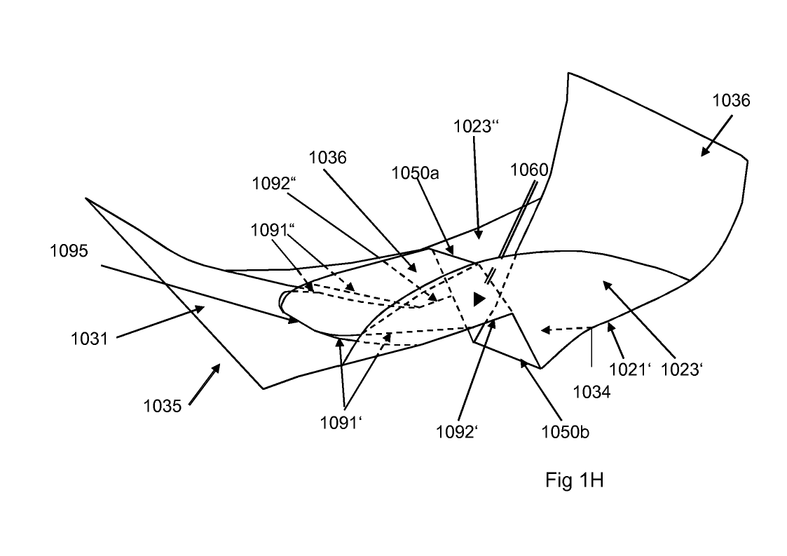

This refers to a design, wherein the side portions of the composite comprising topsheet and backsheet layer and an absorbent core there between, are folded along two predominantly longitudinal fold lines, which may be positioned symmetrically and preferably parallel to the longitudinal centre line, such that portions of the topsheet layer are in a face to face position, such as depicted in FIGS. 1 E and F, which will be explained in more detail herein below. In a particular execution, the folding extends over the full length of the article, though it is sufficient for the present invention, if this folding is at least in a portion of the crotch region. Such a design is also described in WO2011/064275, to which express reference is made.

The overfolding is executed such that the overfolded outer side margins do not overlap, i.e. do not extend beyond the longitudinal centre line, such that a gap having a gap width of preferably at least 10 mm, preferably 25 mm, but less than 100 mm, preferably less than 50 mm is formed.

The overfolding is maintained by a pick-up attachment, as will be explained in more detail herein below. A pick-up attachment does not need to have large geometrical extension and can be executed as a glue dot or a thermo-welded dot, or as a line, a region or a pattern of lines and/or dots. The pick-up attachments may be positioned symmetrically outwardly of the longitudinal centre line between the longitudinal fold lines and the outer side margins of the composite prior to folding, such that upon folding, these pickup attachments are positioned on sections of the topsheet between the product's longitudinal centre line and the fold lines. In case the pickup attachments are positioned between the longitudinal centre line and fold lines prior to folding, they connect to sections between the fold lines and the outer side margins when folded. Pick-up attachments will be positioned in the crotch region of the article, preferably in the region, which corresponds to the crotch line of a wearer during use.

Within the present context, the terms “attached/attachment” relate to the fact, that two or more pieces or elements are connected, preferably permanently connected to each other, such that a force as exerted to one of these is transmitted to others. A particular form of such an attachment can be the use of glues or adhesives, and often a preferred attachment is achieved by thermo-bonding, such as heat, pressure, and/or ultra-sonic welding. Within the present context, “permanently attached” elements or pieces can only be destructively separated, whereby either of the elements, or both, or only the attachment means is destroyed or irreversibly changed. Accordingly, the term “unattached” refers to two or more pieces or elements, which may be in direct contact with each other, but which are essentially unconnected and can be readily separated without destruction or damage. In a non-limiting descriptive example, the backsheet layer and the topsheet layers form an envelope for the core by being attached to each other at their side margins such as by adhesive or thermo-bonding. The enveloped core may then be attached or unattached there between. In the first case it may be attached such as by glue spray to the topsheet layer and/or it may be attached to the backsheet layer. It may also be attached in a first region (e.g. the front region), but not in a second region.

Accordingly, a core, a core element or a core section is attached to the topsheet layer, if it is directly attached to the topsheet layer, or if all intermediate materials or layers are attached—at least in the attachment region where forces are to be transmitted. In the context of the invention it is defined to be a first core section if it is attached to the topsheet layer. Accordingly, when attached to the backsheet layer, it is defined to be a second core section.

It should be noted, that any attachment can only be considered for certain geometric extensions. For example, if two layers are attached to each other by well-known glue spray, there will be small areas such as between glue dots or glue lines, which will not be attached. Nonetheless, considering that the functioning of keeping the layers together is satisfied, they are considered to be attached.

In an in-use configuration, the overfolding of the side margins results in that the article further forms leg hoops around the upper thigh of a wearer. This refers to the fact, that the upper thigh is encircled by an essentially flat strip or band of material, which is connected to the crotch region, and optionally to the front and rear regions, of the article. Upon donning of the article, these hoops exert a pull force to the overfolded side margins and in particular to the pick-up attachment regions. This pull force will automatically and inevitably exert an upward (i.e. towards the wearer) lifting force to the topsheet layer in this pick-up connection region.

The present invention provides a passageway for the bodily exudates in the absorbent structure, which exhibits a length projection onto the cross-directional centre line in the cross-direction of the article, and which has a z-y-directional extension. The perimeter of the passage way is determined by edges of a discontinuity separating the first and second section of the core.

In a first execution, the core sections may be sections of a unitary core element, and the passageway may result from a discontinuity, which exhibits a length projection onto the cross-directional centre line, which is larger than zero, but which does not extend over the full cross-directional extension of the core element, such as when a cross-directional cut line or slit extends from the centre line outwardly towards the longitudinal side margins without intersecting these. Such a discontinuity can be readily made when the core is in a flat (pre-use) configuration. The sections of the core elements which are positioned longitudinally adjacently to and opposite of the discontinuity are then referred to as first and second core sections and the respective edges are referred to as first and second edges respectively. The first edge and core section may be positioned forwardly or rearwardly of the discontinuity. Thus, the perimeter of the passageway is then formed by the first and second edges of the discontinuity

Within the present context, a discontinuity such as a cut-line or slit is considered to allow separation of the structure through its full z-directional cross-section, such that the perimeters of the discontinuity may move independently. Such a discontinuity may be achieved by conventional means, such as without any limitation, cutting by a blade and anvil system, a hydrojet system, a laser cutter, or ultrasonic systems.

It should be noted, that the creation of the discontinuity can be achieved in a two- or multi-step process, such as when in a first step the separation at discontinuity is not completely formed, but complete separation is achieved at a later stage. For example, during manufacturing a perforation along a slit line is applied, and upon donning this perforation is completely opened.

The discontinuity may also be the result of combining two or more sub-sections of a discontinuity, which extend into each other. For example, the discontinuity may have two straight sub-sections, which may intersect at a discontinuity connection point positioned for example on the longitudinal centre line, such that angled or chevron shaped discontinuity results. In another example, the discontinuity may have three sub-sections, which intersect in a discontinuity connection point, thusly forming a Y-shaped or T-shaped discontinuity. Of course, a T-shaped discontinuity may be considered to have two sub-sections, whereby one terminates in the mid-pint of the other. In yet a further example, two sub-sections may intersect thereby forming a +-shaped or x-shaped discontinuity—which of course could also be considered to have four sub-sections intersecting at the discontinuity connection point.

In a further execution, the discontinuity which exhibits a length projection onto the cross-directional centre line, which is larger than zero, may result from combining two or more core elements, such as may result from completely severing a core element into a first and a second sub-element along a predominantly cross-directionally extending separation or cut line, or by combining one core sub-element with another core sub-element.

The passageway is further created and opened by spacing the first and the second edges of the discontinuity z-directionally apart. This spacing apart can be achieved by selectively attaching core sections to the z-directionally adjacent top- or backsheet layers whilst leaving other sections unattached. Thus, the first centre core section may be attached to the topsheet layer in the area adjacent to the discontinuity whilst it is left essentially unattached to the backsheet layer in this area, and the second centre core section, positioned longitudinally opposite to the discontinuity may be attached to the backsheet layer in the area adjacent to the discontinuity whilst it is left essentially unattached to the topsheet layer in this area.

The attachment of the respective core elements to the topsheet layer should extend at least in the region of the pick-up glue, e.g. essentially symmetrically and outwardly of the longitudinal centre line and in proximity to the crotch line.

The skilled person will readily recognize that the spacing of the edges of the discontinuity forming the passageway will be induced when changing the article from a pre-use (or flat or flat-folded) configuration to the in-use configuration, such as prior to or during the donning of the article. Then, as described in the above, the forming of the leg hoops around wearer's legs will transmit pull forces to the topsheet layer in the pick-up connection region and then to the respective first centre core element attached in the respective region. As e.g. this first centre core element is not attached in this region to the backsheet layer, no resistance to this lifting will come from there. As the pull forces from the leg hoop will pull the backsheet layer outwardly—and hence also the e.g. second core element attached thereto—the passageway through the core is fully opened. At the same time the lifting of the overfolded regions will create curvilinear bending lines in the article around the legs, delimiting in the crotch region of the article a centre crotch region from the side crotch regions.

Depending on the intended use of the article, the passageway may have a different orientation.

In a first execution, the first centre core section as attached to the topsheet is positioned forwardly of the discontinuity and the second centre core section as attached to the backsheet is positioned rearwardly of the discontinuity. When the discontinuity is positioned in the crotch region between the anus and urine or menses emanating organs (i.e. penis respectively urethra or vagina), faeces may pass through the passageway and contact the first, forward core section on its outwardly oriented surface, such that the wearer oriented surface remains free for receiving urine or menses.

In a second execution, the first centre core section as attached to the topsheet is positioned rearwardly of the discontinuity and the second centre core section as attached to the backsheet is positioned forwardly of the discontinuity. This execution may be of particular advantage for a user being primarily urine incontinent and often in a reclined position. Then urine loading not absorbed by the front core section will run into the passageway and then be absorbed from the outwardly oriented surface of the rear core section, thusly leaving the wearer oriented surface in a much drier condition as compared to using a single continuous core.

In order to accommodate for the relative movement of the core to the topsheet layer and to the backsheet layer, the latter may be not attached over the full surface to the core. Optionally, these layers may be designed extendible, such as with a fold therein, or by being at least partly elasticised. In particular, when the absorbent core has a certain stiffness and/or thickness, it can be advantageous to apply a second and a third discontinuity in the structure. In order to better conform to the body contours of a wearer, it is a known approach to introduce bending lines in the absorbent structure. However, in order to not compromise on the liquid handling performance and/or core integrity, a complete separation had been avoided in conventional designs.

Applicant has now found that in the context of the present invention and upon consideration of certain design principles, introducing an additional discontinuity may enhance body conformity without any or at least without major detrimental effects on liquid handling or integrity performance.

To this end, one or more further discontinuities may be introduced. The further discontinuity or discontinuities are preferably positioned such that at least one end point of the further discontinuity or discontinuities is longitudinally opposite of the first discontinuity relative to the crotch line. The further discontinuity or discontinuities may have any orientation, but they preferably exhibit a length projection onto the cross-directional centre line, which is larger than zero. The discontinuity or discontinuities should not extend into any of the side margins of the absorbent structure, nor into the first discontinuity.

The discontinuity or discontinuities should preferably be essentially symmetrical to the longitudinally extending centre line of the absorbent structure. Two or more discontinuities may extend into each other. Discontinuities such as slits may advantageously support the forming of a bulge, which further supports the creating and sustaining of the passageway.

### EXEMPLARY EMBODIMENTS

A particular exemplary execution of the present invention is depicted in FIG. 1 A to H.

In FIG. 1A a top view of an absorbent article 1000 in its flat, unfolded and pre-use configuration is schematically depicted, showing a front (1012), a rear (1018) and a crotch (1015) region and a longitudinally extending centre line (1002) and a cross-directionally extending crotch line (1004). In this unfolded configuration, the article shows as first (1010′) and a second (1010″) longitudinally extending side margin. Also indicated are curvilinear bending lines 1021′ and 1021″, demarcating a centre crotch region of the article 1025 and two side crotch regions 1023′ and 1023″, respectively. As can also be seen in the cross-sectional view CC along the longitudinal centre line in FIG. 1B, the article comprises a topsheet layer 1032, attached to a backsheet layer 1037 such as by a construction glue as indicated by “x” 1039 and an absorbent core structure 1035 enveloped by the backsheet and topsheet layers.

A first discontinuity such as a slit line is positioned rearwardly of the cross-directionally extending crotch line 1004, here shown as a cross-directionally extending straight slit line 1050.

FIG. 1C depicts a cross-sectional view along line AA, which is positioned rearwardly of the crotch line, showing the core 1035 attached in the centre crotch region to the backsheet layer 1037 by a first discontinuity attachment indicated by “x” 1052, but essentially unattached to the topsheet layer 1032 as indicated by “o” 1055.

FIG. 1D depicts a cross-sectional view along line BB, which is positioned forwardly of the crotch line, showing the core 1035 attached in the centre crotch region to the topsheet layer 1032 by a second discontinuity attachment indicated by “x” 1057, but essentially unattached to the backsheet layer 1037 as indicated by “o” 1059.

Whilst in FIGS. 1C and 1D the article is shown in a pre-use configuration, FIGS. 1E and 1F show a cross-sectional view of an article now being folded along two longitudinally extending fold lines 1070′ and 1070″, such that the topsheet layer is overlying itself at least in the crotch region 1025. The folding is such that there is a gap 1075 having a cross-directionally extending gap width 1076 between the overfolded side margins 1010′ and 1010″.

Also shown are the first (1083′) and second (1083″) pick-up attachments by which the surfaces of the topsheet layer are attached at least in the crotch region. One or more of such pickup connections may be positioned per side, preferably at different distances to the longitudinal and cross directional article centre lines. Whilst these may extend over a certain area, this region can be relatively small and henceforth these attachments may also be referred to as pick-up points.

When the article is put into its in-use configuration, the front and rear regions are affixed around the waist of the wearer, such as by closure tapes (not shown). The crotch region of the article is positioned in the crotch region of the wearer such that the side crotch regions 1023′ and 1023″ fold along curved fold lines 1021′ and 1021″ respectively such that they lie with their backsheet layer against the inner upper thigh of a wearer. The closure means (not shown) connecting the front and rear regions close the leg hoop, which is now smoothly encircling the leg of the wearer along bending lines 1021.

By the closing of the leg hoops a tension is created in the crotch region, which has different effects forwardly and rearwardly of the discontinuity 1050.

In the forwardly positioned core section, the now overfolded side margins 1010′ and 1010″ are pulled laterally outwardly and upwardly. Consequently, the pick-up attachment will also transmit these forces to the core and the core will be lifted in this region. This is indicated in FIG. 1G showing an enlarged section of FIG. 1B but in an in-use configuration with a lifted core section 1036 attached to topsheet layer 1032 by pick-up attachment 1083′ and 1083″ and being essentially unattached to the backsheet layer 1037 (as indicated by “o” 1059).

In the rearwardly positioned core section, the tacked down core section 1034 is attached to the backsheet layer 1037 an essentially unattached to the topsheet layer 1032 (as indicated by “o” 1055).

Henceforth, passage way 1060 will be created. Thus, if faeces are deposited in the rear section or the article, these may—such as upon gravity or pressure—move down—and forwardly into and through the passageway. The faeces are directed away from the skin of the wearer. In addition, the wearer oriented surface of the absorbent core in the front region is kept free from faecal matter, which otherwise might obstruct the urine handling. Also shown in FIG. 1A is a particular execution wherein a further pair of discontinuities, like first and second slit lines 1091′ and 1091″ is shown.

Such an execution is particularly beneficial, if the core structure 1035 exhibits a certain stiffness. Then introducing the slits will allow bending of the core sections, here shown as the core section 1036 positioned forwardly of the first discontinuity, may in the in-use configuration bulge upwardly laterally inwardly of the slits 1091′ and 1091″ by following the upward lifting forces as described above. Laterally outwardly of the slit lines 1091′ and 1091″ the core will not bulge upwardly. To this end, it is important, that the second discontinuities will not run into the first discontinuity. As a consequence, side bend lines 1092′ and 1092″, which will connect the respective discontinuities, will form and support the bulging. Also, a front bend line will 1095 will form.

FIG. 1H shows schematically the core element of such an article in an in-use configuration. For simplicity, the core element 1035 with its front (1031) and rear (1038) portions is shown as a sheet (i.e. its thickness is neglected). It has a first core section 1036 adjacent to the forward perimeter 1050a of the discontinuity lifted upwardly by the attachment to the topsheet layer (not shown). The second core section 1034 is pulled downwardly by being attached to the backsheet layer (not shown). In FIG. 1H, this section is partially hidden by a side crotch region 1023′. Thus, the passageway 1060 is formed, delimited by the perimeter 1050a and 1050b of the first discontinuity, which transitions into the side crotch regions 1023′ and 1023″. Two further curved discontinuities 1091′ and 1091″ are extending curvilinearly from the front bending line 1095 to side bend lines 1092′ and 1092″. Each of these discontinuities is gapping, here shown by two lines 1091′ and 1091″ respectively. When faecal matter is deposited in the rear portion, it may pass through the passageway 1060 underneath, i.e. away from the skin of the wearer and from the wearer oriented surface of the front portion of the core, leaving this surface free from faecal matter, thusly avoiding impediment of the urine handling capability of the core.

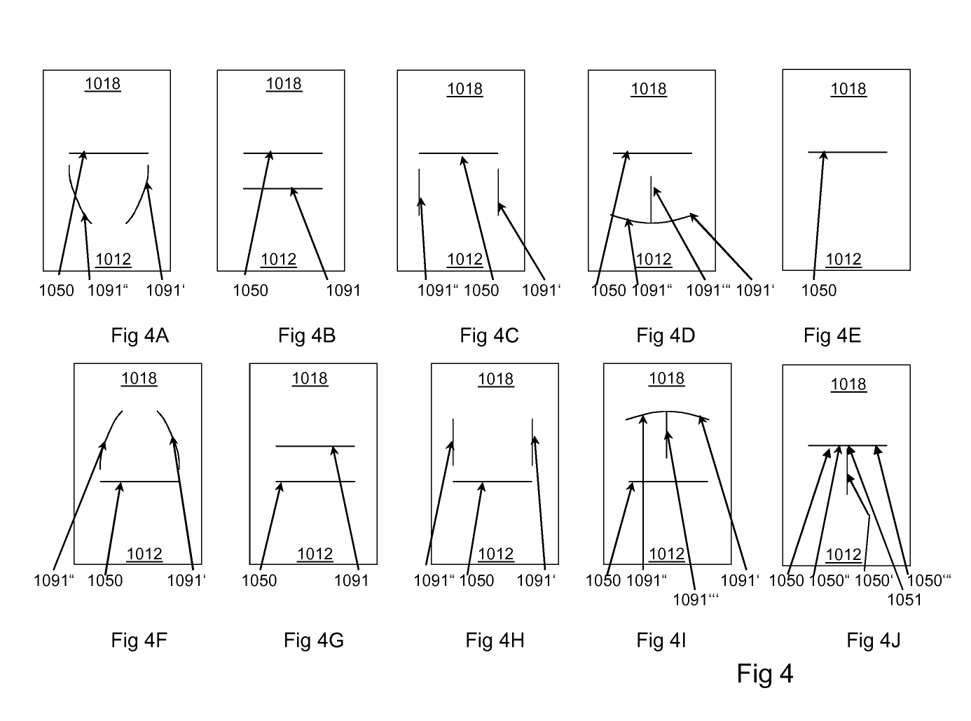

FIG. 4 A to I exemplify non-limiting further options for executing first (1050) and further discontinuities 1091′, 1091″, or 1091′″, especially with their positioning relative to the front (1012) respective rear parts (1018) of the article. FIG. 4J exemplifies a first discontinuity 1050 having several sub-sections 1050′, 1050″, and 1050′″ respectively, which are intercepting, respectively connect at connection point 1051.

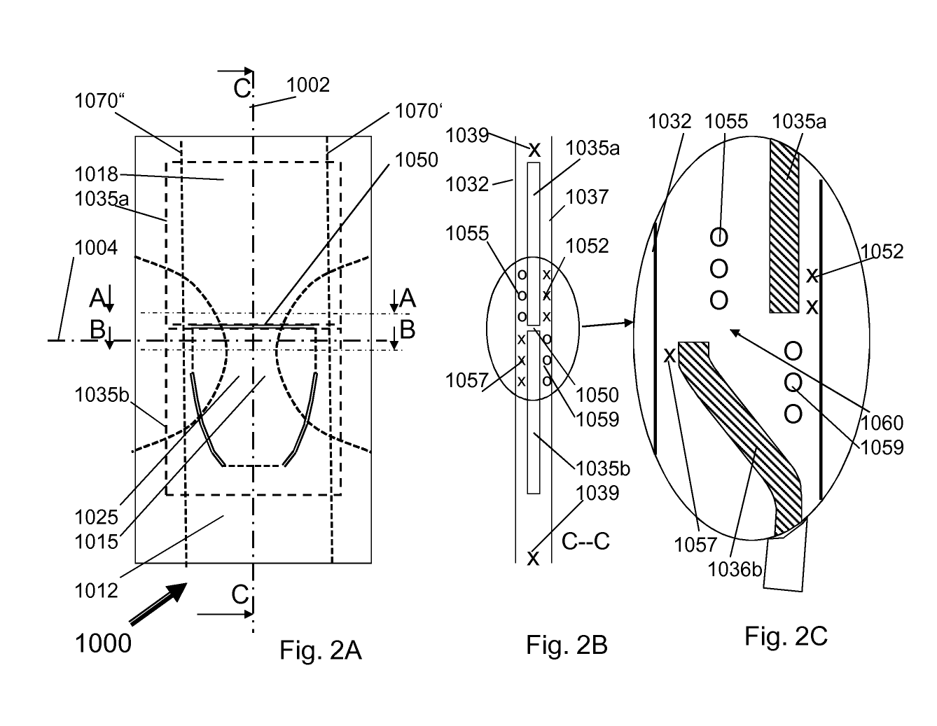

FIG. 2 schematically describes a further particular execution with a non-unitary absorbent core structure but wherein the core structure comprises a first core element 1035a and a second core element 1035b. In FIG. 2C, the latter is shown as 1036b in a lifted configuration, such that the discontinuity 1050 is essentially formed by the margins of the two elements. Optionally, there might be a certain gap between the two core elements.

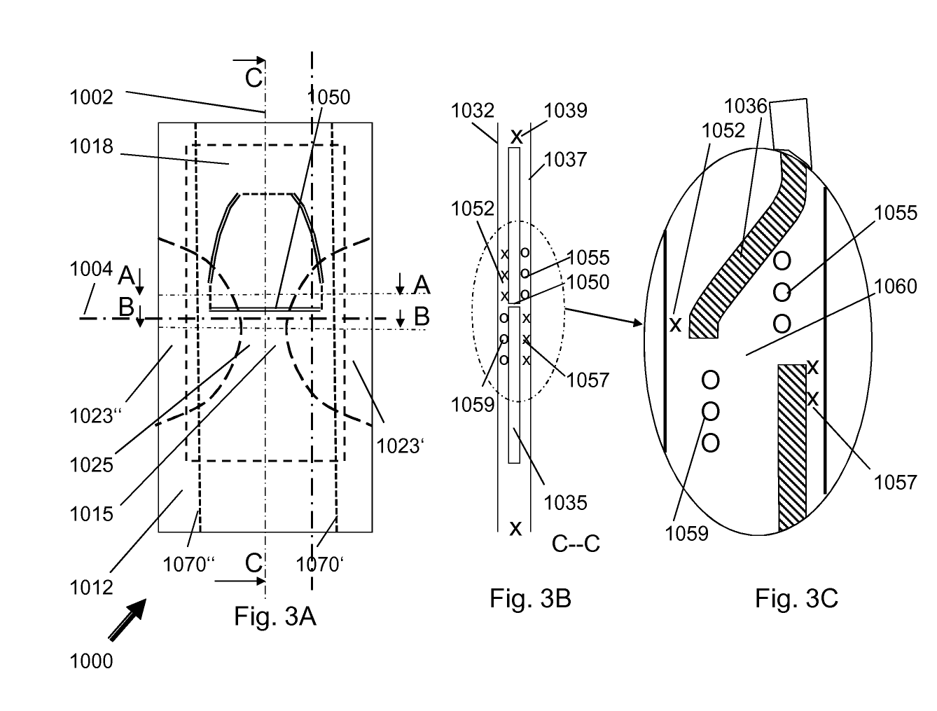

FIG. 3 depicts schematically an execution, which may be particularly effective in handling urinary incontinence for a person in a reclined position. To this end, the orientation of the passageway as described in the above in the context of FIG. 1 is essentially mirrored such that now the section rearwardly of the discontinuity is bulging upwardly. Thus, the passageway 1060 is formed between the rear core section, which is upwardly lifted by being connected to the topsheet layer 1032, and the forward core section which remains connected to the backsheet layer 1037.

1. (canceled)

2. (canceled)

3. (canceled)

4. (canceled)

5. (canceled)

6. (canceled)

7. An absorbent article for being worn on the lower torso of a wearer,
the article comprising
a) a topsheet layer adapted to be in contact with a wearer during its intended use;
b) a backsheet layer positioned opposite to said topsheet away from a wearer during its intended use;
c) an absorbent structure enveloped there between, comprising a front, rear and crotch region thereby defining a longitudinal orientation of the article and the structure and a cross-directional orientation perpendicular thereto, further comprising a cross-directionally extending crotch line for being positioned in the crotch of the wearer;
d) two predominantly longitudinally extending side margins, which are folded over at least in a portion of the crotch region;
e) a pick-up attachment for attaching said overfolded portions of said topsheet at least in a portion of the crotch region to said topsheet;
wherein in said crotch region
i) said absorbent structure comprises a first discontinuity exhibiting a length projection onto the cross-directional centre line, which is larger than zero and separating in the longitudinal direction a first and a second centre core section along a discontinuity line;
ii) at least a portion of said first centre core section is attached to said topsheet-layer and essentially unattached to said backsheet layer at least along the longitudinal centre line, and
iii) at least a portion of said second centre core section is attached to said backsheet layer—and essentially unattached to said topsheet layer at least along said longitudinal centre line,
such that in an in use configuration said topsheet layer and the first centre core section attached thereto are lifted towards the wearer whilst said backsheet layer and said second centre core section attached thereto are pulled into the opposite direction,
such that said discontinuity forms a passageway for allowing bodily exudates to pass through.

8. An absorbent article according to claim 7, wherein said discontinuity extends over the full CD width of the absorbent structure.

9. An absorbent article according to claim 7, wherein said discontinuity does not extend into said longitudinally extending side margins of said absorbent structure, and wherein the cross-directional extension of said discontinuity and said respective attachment respectively unattachment regions is at least 1 cm, but less than 100% of the cross-directional width of the absorbent structure in the region of the discontinuity

10. An absorbent article according to claim 9, wherein the cross-directional extension of said discontinuity and said respective attachment respectively unattachment regions is at least 2 cm.

11. An absorbent article according to claim 7, further comprising at least one further discontinuity,
1) not extending into said side margins of the absorbent structure;
2) not extending into said first discontinuity.

12. An absorbent article according to claim 11, wherein said at least one further discontinuity
3) exhibits a length projection onto the cross-directional centre line, which is larger than zero.

13. An article according to claim 11, comprising at least two further discontinuities which are essentially symmetrical to the longitudinal centre line of the absorbent structure.

14. An article according to claim 11, wherein said two further discontinuities which are essentially symmetrical to the longitudinal centre line of the absorbent structure extend into each other.
